(19) United States
(12) Patent Application Publication
(10) Pub. No.: US 2006/0147457 A1
(43) Pub. Date: Jul. 6, 2006

(54) Purified composition of bispecific molecules and methods of production

(76) Inventors: James P Porter, Sparta, NJ (US); Renay A Ebelle, Hamburg, NJ (US); Leslie Casey, New York, NY (US); Nadya Lukacher, Staten Island, NY (US)

Correspondence Address: JONES DAY, 222 EAST 41ST ST, NEW YORK, NY, 10017, US

(21) Appl. No.: 10/514,547

(22) PCT Filed: May 13, 2003
(86) PCT No.: PCT/US03/15360

§ 371 Date: Feb. 3, 2006

Related U.S. Application Data
(60) Provisional application No. 60/380,211, filed on May 13, 2002.

(51) Int. Cl. A61K 39/395; C07H 21/02

(52) U.S. Cl. 424/178.1; 536/23.1

(57) ABSTRACT

The invention provides a method for producing purified compositions of bispecific molecules each comprising (a) an antigen recognition portion that binds a C3b-like receptor and (b) one or more double-stranded DNA molecules cross-linked to the antigen recognition portion. In particular, the invention provides a method for purifying bispecific molecules using alcohol precipitation. The invention also relates to the purified products of bispecific molecules. The purified compositions of the invention are useful for treating Systemic Lupus Erythematosus (SLE).

This application claims the benefit under 35 U.S.C. § 119(e) of U.S. Provisional Patent Application No. 60/380,211, filed on May 13, 2002, which is incorporated by reference herein in its entirety.

### 1. FIELD OF THE INVENTION

The invention relates to a method of producing purified compositions of bispecific molecules each comprising (a) an antigen recognition portion that binds a C3b-like receptor and (b) one or more double-stranded DNA molecules cross-linked to the antigen recognition portion. The invention also relates to the purified compositions of such bispecific molecules.

### 2. BACKGROUND OF THE INVENTION

Primate erythrocytes, or red blood cells (RBC's), play an essential role in the clearance of antigens from the circulatory system. The formation of an immune complex in the circulatory system activates the complement factor C3b in primates and leads to the binding of C3b to the immune complex. The C3b/immune complex then binds to the type 1 complement receptor (CR1), a C3b receptor, expressed on the surface of erythrocytes via the C3b molecule attached to the immune complex. The immune complex is then chaperoned by the erythrocyte to the reticuloendothelial system (RES) in the liver and spleen for neutralization. The RES cells, most notably the fixed-tissue macrophages in the liver called Kupffer cells, recognize the C3b/immune complex and break this complex from the RBC by severing the C3b receptor-RBC junction, producing a liberated erythrocyte and a C3b/immune complex which is then engulfed by the Kupffer cells and is completely destroyed within subcellular organelles of the Kupffer cells. This pathogen clearance process, however, is complement-dependent, i.e., confined to immune complexes recognized by the C3b receptor, and is ineffective in removing immune complexes which are not recognized by the C3b receptor.

Taylor et al. have discovered a complement independent method of removing pathogens from the circulatory system. Taylor et al. have shown that chemical crosslinking of a first monoclonal antibody (mAb) specific to a primate C3b receptor to a second monoclonal antibody specific to a pathogenic antigenic molecule creates a bispecific heteropolymeric antibody (HP) which offers a mechanism for binding a pathogenic antigenic molecule to a primate's C3b receptor without complement activation. (U.S. Pat. Nos. 5,487,890; 5,470,570; and 5,879,679). Taylor also reported a HP which can be used to remove a pathogenic antigen specific autoantibody from the circulation. Such a HP, also referred to as an “Antigen-based Heteropolymer” (AHP), contains a CR1 specific monoclonal antibody cross-linked to an antigen (see, e.g., U.S. Pat. No. 5,879,679; Lindorfer, et al., 2001, Immunol Rev. 183: 10-24; Lindorfer, et al., 2001, J Immunol Methods 248: 125-138; Ferguson, et. al., 1995, Arthritis Rheum 38: 190-200). In addition to HP and AHP, other methods of producing bispecific molecules having a first antigen recognition domain which binds a C3b-like receptor e.g., a complement receptor 1 (CR1) and a second antigen recognition domain which binds a antigen are also reported (see, e.g., U.S. Provisional application No. 60/276,200, filed Mar. 15, 2001 and No. 60/244,811, filed Nov. 1, 2000; PCT publication WO 01/80883).

Systemic Lupus Erythematosus (SLE) is a complex autoimmune disease that involves defects in regulation of both humoral and cellular aspects of the immune systems. Lupus is characterized by autoantibodies having a spectrum of specificities to nuclear and cytoplasmic antigens (see, e.g., Gauthier et al., 1997, p. 207 In: D. J. Wallace and B. H. Hahn (Eds.), Dubois' Lupus Erythematosus 5^{th} ed. Williams and Wilkins, Baltimore; Stollar, 1981, Clinics Immunil Allergy 1: 243; Tan et al., 1966, J Clin Invest 45: 1732-1740; Tan et al., 1989, Adv Immunol 44: 93-151; Winfield et al., 1977, J Clin Invest 59: 90-96). In particular the presence of anti-dsDNA antibodies is virtually diagnostic of SLE and rarely occurs in other conditions (see, e.g., Hecht et al., 1976, Medicine 55:163-181). High avidity IgG autoantibodies to dsDNA tend to correlate with disease activity (see, e.g., Bootsma et. al., 1997, Ann Rheum Dis 56: 661-666; Emlen et al., 1990, J Immunol Meth 132: 91-101; Swaak et al., 1979, Arthritis Rheum 22: 226-235; Swaak et al., 1986, Ann Rheum Dis 45: 359-366). These antibodies are believed to play a major role in the pathogenesis of SLE, particularly lupus nephritis, through their ability to form immune deposits in the kidney (see, e.g., Aarden et al., 1976, J Immunol Meth 11: 153-163; Aarden et al., 1976, J Immunol Meth 10: 39-48; Emlen; 1986, Arthritis Rheum 29: 1417-1426; Miniter et al., 1979, Arthritis Rheum 22: 959-968; Ebling et al., 1980, Arthritis and Rheumatism 23: 392-403; Vlahakos et al., 1992, Kidney Int. 41: 1690-1700; Gilkeson et al., 1995, Clinical immunology Immunopathol. 76: 59-67; Koffler et al., 1974, Am J Pathol 74: 109-124; Krishnan et al., 1967, J Clin Invest 46: 569-579). This results in cellular proliferation, inflammation and fibrosis leading in some patients to renal failure (see, e.g., Glassock et al., 1991, pp. 1280-1368, In: Brenner, B. M., Rector, F. C. (eds): The kidney Vol. 1 Of 2, W B Saunders, Philadelphia; Koffler et al., 1967, J exp Med 126: 607-624). Administration of anti-DNA antibodies into non-autoimmune mice has been shown to produce nephritis, and transgenic mice expressing a secreted form of an anti-DNA antibody develop lupus nephritis (see, e.g., Vlahakos et al., 1992, Kidney Int. 41: 1690-1700; Gilkeson et al., 1995, Clinical immunology Immunopathol. 76: 59-67; Tsao et al., 1992, J Immunol 140: 350-358).

An AHP based immunotherapy which allows rapid and selective removal of anti-dsDNA antibodies from the circulation of patients with SLE was reported (see, e.g., U.S. Pat. No. 5,879,679; Lindorfer et al., 2001, Immunol Rev. 183: 10-24; Lindorfer, et al., 2001, J Immunol Methods 248: 125-138; Ferguson, et. al., 1995, Arthritis Rheum 38: 190-200; Ferguson, et. al., 1995, J Immunol 38:339-347; Craig et. al., 2000, Arthritis Rheum 43:2265-2275; Emlen et. al., 1986, Arthritis Rheum 29:1417-1425; Edberg et al., 1986, J Immunol 136:4582-87; Taylor et al., Proc. Natl. Acad. Sci. USA 88:3305-3309). Methods employing thiol-activated dsDNA with maleimide-activated antibody for cross-linking dsDNA to antibody have been reported (see, e.g., Gosh et al. 1990, Bioconjugate Chemistry 1: 71-76; Lindorfer, et al., 2001, J Immunol Methods 248: 125-138). In Gosh (Gosh et al. 1990, Bioconjugate Chemistry 1: 71-76), a 70% yield on the thiol derivatization of oligonucleotides was reported. Gosh also reported using a molar excess of 50:1 for the maleimide derivitization step and reported that the resulted derivatized alkaline phosphatase (140 kD) contained 6 maleimide residues. Gosh reported that a 5:1 (input protein to oligo ratio) yielded a conjugate with a one to one component composition. Lindorfer's conjugation protocol used a molar excess of 9:1 for the maleimide derivitization step (Lindorfer et al., 2001, J Immunol Methods 248: 125-138). In Lindorfer, various molar ratios of the input antibody 7G9 IgG to dsDNA were used for the synthesis of AHP.

There is therefore a need for AHP or bispecific molecule products that are safe and have efficacy for treatment of SLE in patients. There is a need for improved methods and processes for production of bispecific molecules or AHPs containing an antibody cross-linked to a dsDNA. There is especially a need for more efficient methods for purifying and/or concentrating bispecific molecule or AHP products.

Discussion or citation of a reference herein shall not be construed as an admission that such reference is prior art to the present invention.

### 3. SUMMARY OF THE INVENTION

The present invention provides methods for producing purified compositions of bispecific molecules each comprising (a) an antigen recognition portion that binds a C3b-like receptor and (b) one or more double-stranded DNA molecules cross-linked to the antigen recognition portion. The method of the invention comprises precipitating the bispecific molecules using an alcohol solution, e.g., a 50% (volume) isopropyl alcohol solution. In a preferred embodiment of the invention, the method comprises (i) cross-linking an antigen recognition portion with one or more dsDNA molecules to produce a composition comprising bispecific molecules comprising the antigen recognition portion cross-linked to one or more of the dsDNA molecules; and (ii) precipitating the composition using an alcohol solution, e.g., a 50% (volume) isopropyl alcohol solution, to produce the purified composition.

In a specific embodiment of the invention, the method comprises (i) reacting dsDNA molecules with 1-Ethyl-3-(3-dimethylaminopropyl)-carbodiimide to produce activated phosphorimidazolide-dsDNA (PI-dsDNA); (ii) reacting the PI-dsDNA with cystamine to produce cystaminated dsDNA; (iii) reacting the cystaminated dsDNA with dithiothreitol to produce SH-dsDNA; (iv) reacting the antigen recognition portion with sulfosuccinimidyl 4-(N-maleimidomethyl) cyclohexane-1-carboxylate to produce maleimide modified antigen recognition portion; (v) reacting the SH-dsDNA with the maleimide modified antigen recognition portion to produce a composition comprising bispecific molecules comprising the antigen recognition portion cross-linked to the one or more dsDNA molecules; and (vi) precipitating the composition using an alcohol solution, e.g., a 50% (volume) isopropyl alcohol solution, to produce the purified composition. In a preferred embodiment, the method further comprises before step (vi) a step of removing free IgG antibodies from the composition produced in step (v) using ion exchange chromatography.

In any one of the methods of the invention, the antigen recognition portion that binds a C3b-like receptor is preferably an anti-CR1 monoclonal antibody, e.g., a 7G9 monoclonal antibody. Preferably, the dsDNA molecules used in the method of the present invention have an average base pair size in the range of 100 to 5000. More preferably, the DNA molecules have a size in the range of 200 to 3000 base pairs. Still more preferably, the DNA molecules have a size in the range of 500 to 2500 base pairs. Still more preferably, the DNA molecules have a size in the range of 500 to 1500 base pairs. In a specific embodiment, the DNA molecules have an average size of 2100 base pairs.

The invention also provides purified compositions of bispecific molecules as produced by the method of the invention. In a preferred embodiment, the DNA concentration of the purified composition is at least 1.100 mg/ml. In another preferred embodiment, the protein concentration of the purified composition is at least 0.200 mg/ml. In still another preferred embodiment, the purified composition has a titer of at least 0.030 mg/ml as determined by ELISA with immobilized CR1 receptors. In still another preferred embodiment, the purified composition has a free IgG protein concentration of <0.006 mg/ml.

The bispecific molecule of the invention can be used to remove anti-DNA antibody from the circulation of patients with SLE, and therefore is useful for treating SLE.

### 4. BRIEF DESCRIPTION OF FIGURES

FIG. 1 is a schematic illustration of an exemplary conjugation process for producing a bispecific molecule comprising a anti-CR1 mAb cross-linked to a double-stranded DNA molecule.

FIG. 2 is a schematic illustration of an exemplary purification process for producing a bispecific molecule comprising a anti-CR1 mAb cross-linked to one or more double-stranded DNA molecules.

FIG. 3 Timeline (in minutes) of events in monkey protocols. Monkeys not receiving AHP were infused saline during the 35-37 min interval. Blood and plasma samples were collected at the indicated time points to monitor AHP binding to erythrocytes and to follow human anti-dsDNA antibody clearance. Blood and serum chemistries were assayed on samples withdrawn at T=0, T=35, T=97, T=277 minutes and on day 1.

FIGS. 4A-4C illustrate detection of AHP binding to monkey red blood cells. Suspensions of 1.4×10⁸ E/ml were incubated (in duplicate) for 30 min with probes specific for the dsDNA component Pico Green) or CR1 mAb component (Alexa anti-mouse IgG) of AHP. Data reported are the average of the mean fluorescence intensity (MFL) of the stained samples subtracting the average mean fluorescence intensity of non-stained duplicate samples (Specific MFL). Monkeys used for data shown in 4A and 4B were infused with 1.5 mg AHP (cGMP material ET104). 4C shows the results for one of two monkeys infused with saline.

FIGS. 5A-5B Detection of Human anti-dsDNA antibody binding to monkey red blood cells. Suspension of 1.4×10⁸ E/ml were incubated (in duplicate) for 30 min with a mixture of probes specific for human IgG and IgM proteins. Data reported are the average of the mean fluorescence intensity of the stained samples subtracting the average mean fluorescence intensity of non-stained duplicate samples. Monkeys used for data shown in SA were infused with 1.5 mg AHP (cGMP material ET104). 5B shows the results for two monkeys infused with saline.

FIGS. 6A-6D Detection of Human anti-dsDNA antibody in Plasma from infused monkeys. Panels A and B report the level of high affinity anti-dsDNA antibody (Farr activity) present in plasma from monkeys infused with AHP (FIG. 6A) or saline (FIG. 6B). FIGS. 6C and 6D detect the level of Human anti-dsDNA antibody (human Ig) in plasma capable of binding to monkey erythrocyte that contain AHP on their surface. Suspensions of naive monkey RBC (1.4×10⁹E/ml) were opsonized by incubating for 15 min with a saturating amount of (3 μg/ml) AHP. After washing the sample was resuspended to ˜1% E and 50 μl of the opsonized erythrocyte was incubated for 15 min with 50 μl of infused monkey plasma. After washing, the sample was stained for 30 min with a mixture of Alexa anti-human IgG and anti-human IgM reagent. Data reported are the average of the mean fluorescence intensity of the stained samples subtracting the average mean fluorescence intensity of non-stained duplicate samples. Monkeys used for data shown in FIG. 6A were infused with 1.5 mg AHP (cGMP material ET104). FIG. 6B shows the results for two monkeys infused with saline.

### 5. DETAILED DESCRIPTION OF THE INVENTION

The present invention provides methods for producing purified compositions of bispecific molecules each comprising an antigen recognition portion that binds a C3b-like receptor cross-linked to one or more double-stranded DNA molecules. As used herein, the term “C3b-like receptor” refers to any mammalian circulatory molecule expressed on the surface of a mammalian blood cell, which has an analogous function to a primate C3b receptor, the CR1, in that it binds to a molecule associated with an immune complex, which is then chaperoned by the blood cell to, e.g., a phagocytic cell for clearance. In the disclosure, anti-CR1 portion or anti-CR1 antibody is often referred to. It will be apparent to one skilled person in the art that the disclosure is equally applicable to antigen recognition portion that binds other C3b-like receptors. A mammalian blood cell can be, but is not limited to, a primate red-blood cell or erythrocyte. The invention also relates to the purified compositions of such bispecific molecules.

The bispecific molecule of the invention can be used to remove anti-DNA antibody from the circulation of patients with SLE, and therefore is useful for treating SLE. The bispecific molecule of the invention is a type of antigen-based heteropolymer (AHP). This technology makes use of the immune adherence function of primate erythrocyte (hereinafter also referred to as “E”) to design and develop therapeutic products for targeting and clearing pathogens in the bloodstream. The AHP heteropolymer of the present invention can contain a mAb specific for CR1 chemically crosslinked to dsDNA. E-bound AHP captures dsDNA autoantibodies as E-bound immune complexes. The E-bound immune-complexes, when delivered to the liver, is acted upon by fixed tissue macrophages, resulting in removal of the CR1-AHP dsDNA Ab complexes for endocytosis and return of the E to the circulation.

### 5.1. Production of Anti-CR1 Portion

In preferred embodiments, the anti-CR1 portion of the bispecific molecule comprises an anti-CR1 mAb. An anti-CR1 mAb that binds a human C3b receptor can be produced by known methods. In one embodiment, anti-CR1 mAb, preferably an anti-CR1 IgG, can be prepared using standard hybridoma precedure known in the art (see, for example, Kohler and Milstein, 1975, Nature 256:495-497; Hogg et al., 1984, Eur. J. Immunol. 14:236-243; O'Shea et al., 1985, J. Immunol. 134:2580-2587; Schreiber, U.S. Pat. No. 4,672,044). A suitable mouse is immunized with human CR1 which can be purified from human erythrocytes. The spleen cells obtained from the immunized mouse are fused with an immortal mouse myeloma cell line which results in a population of hybridoma cells, including a hybridoma that produces an anti-CR1 antibody. The hybridoma which produces the anti-CR1 antibody is then selected, or ‘cloned’, from the population of hybridomas using conventional techniques such as enzyme linked immunosorbent assays (ELISA). Hybridoma cell lines expressing anti-CR1 mAb can also be obtained from various sources, for example, the murine anti-CR1 mAb that binds human CR1 described in U.S. Pat. No. 4,672,044 is available as hybridoma cell line ATCC HB 8592 from the American Type Culture Collection (ATCC). The obtained hybridoma cells are grown and washed using standard methods known in the art. Anti-CR1 antibodies are then recovered from supernatants.

In other embodiments, nucleic acids encoding the heavy and light chains of an anti-CR1 mAb, preferably an anti-CR1 IgG, are prepared from the hybridoma cell line by standard methods known in the art. As a non-limiting example, cDNAs encoding the heavy and light chains of the anti-CR1 IgG are prepared by priming mRNA using appropriate primers, followed by PCR amplification using appropriate forward and reverse primers. Any commercially available kits for cDNA synthesis can be used. The nucleic acids are used in the construction of expression vector(s). The expression vector(s) are transfected into a suitable host. Non-limiting examples include E. coli, yeast, insect cell, and mammalian systems, such as a Chinese hamster ovary cell line. Antibody production can be induced by standard method known in the art.

In another embodiments, anti-CR1 scFv's are prepared according to standard methods known in the art. In one embodiment, anti-CR1 chimeric antibodies and nucleic acids encoding such anti-CR1 chimeric antibodies are prepared according to standard methods known in the art (see, e.g., U.S. Pat. Nos. 4,816,567, 4,816,397, 5,693,762; 5,585,089; 5,565,332 and 5,821,337 which are incorporated herein by reference in their entirety).

Anti-CR1 antigen recognition portions can also be produced by standard phage display technologies. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

### 5.2. Preparation of dsDNA

Double stranded DNA molecules can be obtained from various sources. In a preferred embodiment, highly polymerized, double-stranded deoxyribonucleic acid isolated from salmon testis (e.g., Sigma, catalogue# D1626) is used. In another preferred embodiment, calf thymus DNA is used. Preferably, dsDNA used in the present invention is produced under cGMP compliance. More preferably, dsDNA used in the present invention is tested for viral adventitious agents, mycoplasma and endotoxins to ensure the absence of these substances.

DNA molecules are preferably of a particular size or particular range of sizes. Preferably, dsDNA molecules used in the present invention have an average base pair size in the range of 100 to 5000. More preferably, the DNA molecules have a size in the range of 200 to 3000 base pairs. Still more preferably, the DNA molecules have a size in the range of 500 to 2500 base pairs. Still more preferably, the DNA molecules have a size in the range of 500 to 1500 base pairs. In a specific embodiment, the DNA molecules have an average size of 2100 base pairs.

In one embodiment, dsDNA used to produce the AHP of the present invention is fragmented from highly polymerized DNA, e.g., salmon sperm DNA. Any methods known in the art can be used for this purpose, including but not limited to sonication. In a specific embodiment, a Misonix 3000 Ultrasonic Liquid processor with a flow through sonication chamber is used to allow fragmentation of large volume of dsDNA. In this embodiment, dried solid dsDNA is suspended in a DNA buffer (e.g., 10 mM Imidazole, 100 mM NaCl, 1 mM EDTA pH 7.5) by mixing at chosen temperature, e.g., 37° C., for a suitable period of time, e.g., 2 hours. The dsDNA suspension is then chilled and subsequently sonicated. Preferably, the dsDNA suspension is chilled to 4-8° C. overnight. The sonication is carried out by circulating the DNA suspension through the sonication chamber at a flow rate of 150 milliliters per minute and a power setting of 7. One-500 milliliter batch of suspended DNA is cycled through the chamber three times. After sonication, each batch can be evaluated for base pair distribution by standard method known in the art, e.g., using an Agilent Bioanalyzer. Preferably, dsDNA molecules are fragmented to a size in the range of 100 to 5000 base pairs. More preferably, DNA molecules are fragmented to a size in the range of 200 to 3000 base pairs. More preferably, DNA molecules are fragmented to a size in the range of 500 to 2500 base pairs. Still more preferably, DNA molecules are fragmented to a size in the range of 500 to 1500 base pairs. In a specific embodiment, DNA molecules are fragmented to an average size of 2100 (estimated MW ˜1,386,000 Daltons). The obtained dsDNA pool can be aliquoted and stored at −20° C.

### 5.3. Production of Bispecific Molecules

The bispecific molecule, or AHP, of the present invention can be a covalent conjugate of one or more dsDNA molecules with a protein having CR1 binding specificity (e.g., an anti-CR1 monoclonal antibody, e.g., the 7G9 antibody as described in U.S. Pat. No. 5,879,679). Any standard chemical cross-linking methods can be used in the present invention. For example, cross-linking agents, including but not limited to, protein A, glutaraldehyde, carbodiimide, N-succinimidyl-S-acetyl-thioacetate (SATA), N-succinimidyl-3-(2-pyridyldithio)propionate (SPDP), and sulfosuccinimidyl 4-(N-maleimidomethyl) cyclohexane-1-carboxylate (sSMCC) can be used. In a preferred embodiment, cross-linking agent sSMCC is used to cross-link an anti-CR1 portion and a dsDNA.

In one embodiment, by way of example but not limitation, the following protocol is used. A suspension of dsDNA of a suibtable size is diluted with stock 1M Buffer (1M Imidazole pH 6.0) to a suitable concentration. In a preferred embodiment, the resulting dsDNA concentration is ˜0.5 mg/ml. 1-Ethyl-3-(3-dimethylaminopropyl)-carbodiimide (Pierce, Cat#22980, hereinafter “EDC”) at a suitable molar ratio to DNA is added to the dsDNA solution in one or more portions. In a preferred embodiment, a molar ratio of 115,000:1 EDC:DNA is added to the dsDNA solution in three equal aliquots at T=0, T=20 and T=40 minutes. The reaction mixture is preferably inverted after the addition of each portion of EDC to dissolve the EDC. The reaction mixture is then allowed to react for a suitable period of time. Preferably, the reaction mixture is held at room temperature and mixed 4-5 times over an 1 hour reaction period. The product of this reaction is an activated phosphorimidazolide-dsDNA (PI-dsDNA).

Preferably, the activated PI-dsDNA is purified using any standard method known in the art, e.g., by alcohol precipitation. In a preferred embodiment, the activated PI-dsDNA reaction mixture is combined with a stock buffer (1M cystamine 50 mM Hepes pH8.2, hereinafter “CYS buffer”) to give a final Cystamine concentration of 0.25M. The reaction is preferably mixed thoroughly by, e.g., inversion. The mixture is then placed for 2 hrs at 40° C. The pH of the mixture is preferably pH 7.5. The reaction mixture is then placed on ice and adjusted to 0.3 M Sodium Acetate by adding cold 3M sodium acetate, pH 5.8 (hereinafter “ACE buffer”). An equal volume of cold isopropyl alcohol is then added and the mixture is incubated on ice for 10 min. The chilled mixture is clarified by centrifuging for 45 min at 2,000 g (Beckman centrifuge J-6B, JS 3.0 rotor at 3200 rpm). The pellet is dried by flushing the tube with argon gas for 10 min The dried pellet (cystaminated DNA) is resuspended in HSPE buffer (50 mM Phosphate, 1M NaCl pH7.6), the suspension is vortexed for 2-3 min, and the sample is incubated at 40° C. for 20 min in a shaking air incubator. The reduction of cystaminated dsDNA to SH-DNA is then carried out, preferably by reaction with dithiothreitol. In a preferred embodiment, the reduction of cystaminated DNA to SH-DNA is carried out as follows: Dithiothreitol (DTT) is added to the cystaminated dsDNA to a final concentration of 100 mM DTT; the suspension is mixed by gentle inversion and the tube flushed with argon gas; the reduction is allowed to continue at room temperature for 60 min with mixing; the SH-dsDNA product is then recovered by two isopropyl alcohol precipitations, dried with argon gas, and resuspended in a buffer (50 mM phosphate 150 mM NaCl 1 mM EDTA pH7.6, hereinafter “PBSE buffer”) by vortexing and incubating at 40° C. for 15 mill in a shaking air incubator. The container with SH-dsDNA is preferably flushed with argon gas, sealed with parafilm. The SH-dsDNA is preferably used within 15 min.

Preferably, the antibody derivatization process is carried out concurrently with the reduction process of the cystaminated-DNA such that both are ready for the crosslinking step simultaneously. The antibody can be derivatized with maleimide using any method known in the art. In one embodiment, the antibody is derivatized with maleimide as follows: a fresh stock solution (7 mM) of sSMCC Conjugation solution is prepared in PBSE buffer; the antibody is dialyzed exhaustively against PBSE buffer; the coupling reaction is initiated by combining the antibody and sSMCC at a molar ratio of 1:9; the reactants are mixed by inversion and incubated at room temperature for 60 min with mixing; and the sSMCC-antibody is recovered by size exclusion chromatography using FPLC with two Pharmacia 26/10 Desalting Columns in series (cat#17-5087-01). The column is preferably pre-washed with distilled water followed by PBSE buffer according to the manufacturer's instructions before loaded with the reaction mixture. The maleimide modified antibody is eluted in the void volume with PBSE buffer and should be used within 15 min.

The maleimide-antibody and SH-dsDNA are then combined at a desired molar ratio of mAb:DNA. In a preferred embodiment, the maleimide-antibody and SH-dsDNA are combined at a molar ratio of 6:1 (mAb:DNA). In one embodiment, the reaction vessel is flushed with argon, sealed with parafilm, covered with foil and the cross-linking reaction allowed to proceed for 15 hrs at room temperature with mixing by rotation.

### 5.4. Purification of Bispecific Molecules

The bispecific molecules produced by a method such as described supra are then preferably purified. In a preferred embodiment, the conjugation reaction mixture obtained in Section 5.3, or a portion thereof, is fractionated on a DEAE-Sepharose ion exchange resin, preferably at pH 7.6. Column is prepared according to instructions from manufacturer. In a preferred embodiment, the DEAE column run is carried out at a flow rate of 5 ml/min. One bulk fraction is collected as the flow passes through. The non-binding portion containing free IgG can be collected at port. The AHP product is eluted from the column. In one embodiment, the AHP product is eluted from the column with 0.5 M NaCl. The product is collected as a bulk.

Preferably, the AHP product is purified by using alcohol precipitation, e.g., Isopropyl Alcohol (IPA) precipitation. While IPA is preferred for use, it is contemplated that other alcohols (e.g., ethanol, isobutyl alcohol) may also be used. In a preferred embodiment, the method comprises first diluting the AHP product in an appropriate buffer, e.g., by adding a suitable amount of cold ACE Buffer to the AHP product, and then adding a suitable amount of cold IPA to the solution. In a specific embodiment, the amounts of buffer and IPA are chosen such that a 50% alcohol mixture by volume is obtained. Preferably, the alcohol mixture is centrifugated and the pellet retained and suspended in an appropriate buffer. In particular, the mixture is preferably centrifugated at 2.3K×g. The pellet is retained and the supernatant is discarded. Preferably, the pellet is further processed as described below: the centrifuge tube is inverted onto a paper towel for 3-5 minutes; the tube is then argon gas dried for 15 minutes; the pellet is suspended in a suitable amount of sterile PBS Buffer, vortexed and incubated at 40° C. for 20 minutes in a shaking air incubator.

### 5.5. Purified Compositions of Bispecific Molecules

The purity and/or concentration of the bispecific molecule composition of the invention can be characterized using any standard method known in the art. In some embodiments, the concentration of the bispecific molecule composition is characterized based on the DNA concentration. In one embodiment, the DNA concentration is determined using a Picogreen assay. In another embodiment, the DNA concentration is determined by measuring UV absorbance. The DNA concentration is determined as the absorbance at 260 nm divided by 20, i.e., A260/20. In preferred embodiments, the bispecific molecule composition produced by the method of the present invention has a DNA concentration of at least 0.600 mg/ml, more preferably at least 0.680 mg/ml, still more preferably at least 0.700 mg/ml, still more preferably at least 0.800 mg/ml, still more preferably at least 0.935 mg/ml, most preferably at least 1.100 mg/ml.

The concentration of the bispecific molecule composition can also be characterized based on the protein concentration. In one embodiment, the protein concentration is determined using a lowry assay. Preferably, the bispecific molecule composition produced by the method of the present invention has a protein concentration of at least 0.100 mg/ml, more preferably at least 0.200 mg/ml, still more preferably at least 0.250 mg/ml, most preferably at least 0.300 mg/ml.

The concentration of the bispecific molecule composition can also be characterized based on the functional activity of the bispecific molecules. In one embodiment, the anti-CR1 binding activity is determined using ELISA with immobilized CR1 receptor molecules (attached to a solid phase, e.g., a microtiter plate). The assay is also referred to as a CR1/Antibody assay or CAA, and can be used generally to measure any anti-CR1 antibody, or HP or AHP containing an anti-CR1 antibody. In a preferred embodiment, ELISA/CR1 plates are prepared by incubating ELISA plates, e.g., high binding flat bottom ELISA plates (Costar EIA/RIA strip plate 2592) with a suitable amount of a bicarbonate solution of CR1 receptors. Preferably, the concentration of the bicarbonate solution of CR1 receptors is 0.2 ug/ml prepared from 5 mg/ml sCR1 receptors stock (Avant Technology Inc.) and a carbonate-bicarbonate buffer (pH 9.6, Sigma C-3041). In a preferred embodiment, 100 ul CR1-bicarbonate solution is dispensed into each well of the ELISA plates and the plates are incubated at 4° C. overnight. The plates are then preferably washed using, e.g., a wash buffer (PBS, 0.1% Tween-20, 0.05% 2-Chloroacetamide). In another preferred embodiment, a SuperBlock Blocking Buffer in PBS (Pierce) is added to the plates for about 30-60 min at room temperature after the wash. The plates can then be dried and stored at 4° C. The titration of anti-CR1 Abs or AHP can be carried out using a CR1 binding protein, e.g., human anti-CR1 IgG, as the calibrator. In a preferred embodiment, the calibrator a human anti-CR1 IgG having a concentration of 300 or 600 mg/ml. In one embodiment, the titration of the purified composition of bispecific molecules of the invention is carried out using PBS, 0.25% BSA, 0.1% Tween-20 as the diluent buffer, PBS, 0.1% Tween-20, 0.05% 2-Chloroacetamide as the wash buffer, TMB-Liquid Substrate System for ELISA (3,3′, 5.5′-Tetramethyl-Benzidine) and 2N H₂SO₄ as the stop solution. Preferably, the bispecific molecule composition produced by the method of the present invention has an CAA titer of at least 0.030 mg/ml, more preferably at least 0.050 mg/ml, still more preferably at least 0.060 mg/ml, still more preferably at least 0.070 mg/ml, and most preferably at least 0.080 mg/ml.

The purity of the bispecific molecule composition of the invention can also be characterized using any standard method known in the art. In one embodiment, high-performance size exclusion chromatography (HPLC-SEC) assay is used to determined the content of contamination by free IgG proteins. In preferred embodiments, the bispecific molecule composition produced by the method of the present invention has a contaminated IgG concentration of less than 0.010 mg/ml, more preferably less than 0.008 mg/ml, most preferably less than 0.006 mg/ml.

### 6. EXAMPLE

The following example describes the production of a bispecific molecule comprising an anti-CR1 mAb and dsDNA.

Production of anti-CR1×dsDNA

A hybridoma cell line secreting a high-affinity anti-CR1 monoclonal antibody, 7G9 (murine IgG2a, kappa), was used. A master cell bank (MCB) was generated from this cell line and tested (Charles River Tektagen) for mouse antibody production, mycoplasma and sterility.

The 7G9 mAb utilized for the production of both preclinical AHP and clinical AHP originated from the same MCB. The 7G9 antibody used in the production of the preclinical AHP (ET051-45C) was produced and purified from ascites fluid. The 7G9 monoclonal antibody used for the production of clinical AHP (ETI 104) was produced in vitro (hollow-fiber bioreactor) and purified under cGMP through a services contract with Goodwin Biotechnology, Inc. (Plantation, Fla.).

Highly polymerized, double-stranded deoxyribonucleic acid (dsDNA) was isolated from salmon testis. The dsDNA for preclinical AHP production, was purchased from Sigma (catalogue# D1626). The dsDNA used in the production of clinical AHP was produced under cGMP compliance through a supply agreement with Sigma-Aldrich (St. Louis, Mo.) and tested for viral adventitious agents, mycoplasma and endotoxins (Charles River Tektagen).

A Misonix 3000 Ultrasonic Liquid processor, with a flow through sonication chamber was used to fragment large volumes of suspended dsDNA. Dried solid dsDNA was suspended in DNA buffer (10 mM Imidazole, 100 mM NaCl, 1 mM EDTA pH 7.5) by mixing at 37° C. for two hours. The dsDNA suspension (2 mg/ml) was chilled to 4-8° C. overnight and subsequently sonicated as follows to fragment the dsDNA. According to the manufacturers suggested protocol, the DNA suspension was circulated through the sonication chamber at a flow rate of 150 milliliters per minute and a power setting of 7. One-500 milliliter batch of suspended DNA was cycled through the chamber three times. Each sonicated batch was evaluated for base pair distribution by analysis with an Agilent Bioanalyzer. All samples had average base pair size of 2100 (estimated MW ˜1,386,000 Daltons) and were combined into one bulk. The dsDNA pool was aliquoted and stored at −20° C.

AHP is a covalent conjugate of dsDNA fragments and a specific monoclonal antibody (7G9). To produce AHP, fragmented dsDNA was cross-linked to anti-CR1 mAb 7G9 in the following manner. One portion of sonicated dsDNA was thawed at room temperature. The dsDNA suspension was diluted with stock 1M Buffer (1M Imidazole pH6.0) to a final concentration of 0.1M Imidazole pH6.0. The resulting dsDNA concentration was ˜0.5 mg/ml. EDC reagent (Pierce, Cat#22980) was thawed at room temperature. EDC (molar ratio 115,000:1 EDC:DNA) was added to the dsDNA solution in three equal aliquots at T=0, T=20 and T=40 minutes. The reaction mixture at each addition was inverted to dissolve the EDC, then held at room temperature and mixed manually 4-5 times over the 1 hour reaction period. The product of this reaction was an activated phosphorimidazolide-dsDNA (PI-dsDNA). The activated PI-dsDNA reaction mixture was combined with stock CYS buffer (1M Cystamine 50 mM Hepes pH8.2) to give a final Cystamine concentration of 0.25M. The reaction was mixed thoroughly by inversion, the pH was checked (pH 7.5) and placed for 2 hrs at 40° C. The reaction mixture was placed on ice and adjusted to 0.3 M Sodium Acetate by adding cold ACE buffer (3M Acetate pH6.0). An equal volume of cold isoporopyl alcohol was added and the mixture was incubated on ice for 10 min. The chilled mixture was clarified by centrifuging for 45 min at 2,000 g (Beckman centrifuge J-6B, JS 3.0 rotor at 3200 rpm). The pellet was dried by flushing the tube with argon gas for 10 min. The dried pellet (cystaminated DNA) was resuspended in HSPE buffer (50 mM Phosphate, 1M NaCl pH7.6), the suspension was vortexed for 2-3 min, and the sample was incubated at 40° C. for 20 min in a shaking air incubator.

The antibody derivatization process occurred concurrently with the reduction process of the cystaminated-DNA such that both were ready for the crosslinking step simultaneously. The following describes the reduction of cytaminated DNA to SH-DNA. Dithiothreitol (DTT) was added to the cystamidate-dsDNA to a final concentration of 100 mM DTT. The suspension was mixed by gentle inversion and the tube flushed with argon gas. The reduction continued at room temperature for 60 min with mixing. The SH-dsDNA product was recovered by two isopropyl alcohol precipitations, dried with argon gas, and resuspended in PBSE buffer (50 mM phosphate 150 mM NaCl 1 mM EDTA pH7.6) by vortexing and incubating at 40° C. for 15 min in a shaking air incubator. The container with SH-dsDNA was flushed with argon gas, sealed with parafilm and used within 15 min.

Antibody was derivatized with maleimide as follows: a fresh stock solution (7 mM) of sSMCC Conjugation solution was prepared in PBSE buffer. The 7G9 anti-CR1antibody was dialyzed exhaustively against PBSE buffer. The coupling reaction was initiated by combining the antibody and sSMCC at a molar ratio of 1:9. The reactants were mixed by inversion and incubated at room temperature for 60 min with mixing. The sSMCC-antibody was recovered by size exclusion chromatography using FPLC with two Pharmacia 26/10 Desalting Columns in series (cat#17-5087-01). The column was pre-washed with distilled water followed by PBSE buffer according to the manufacturer's instructions then loaded with the reaction mixture. The maleimide modified 7G9 was eluted in the void volume with PBSE buffer and used within 15 min.

The Maleimide-7G9 mAb and SH-dsDNA were combined at a molar ratio of 6:1 (mAb:DNA). The reaction vessel was flushed with argon, sealed with parafilm, covered with foil and the cross-linking reaction allowed to proceed for 15 hrs at room temperature with mixing by rotation. The AHP product was purified from the reaction mixture by DEAE Sepharose Ion Exchange Chromatography and eluted from the column with 50 mM Phosphate, 0.5M NaCl, pH7.6. The product was precipitated with isopropyl alcohol, dried with argon gas and resuspended in sterile PBS buffer by vortexing and incubating at 40° C. for 20 min in a shaking air incubator.

Evaluation of 7G9×dsDNA in a Monkey SLE Model

Several mouse models of SLE have been developed, most notably the NZB/NZW F1 hybrids and MRL lpr/lpr mouse strains (Birmingham et al., 2001, Immunol Res 24:211-224). However, in mice CR1 is not present on E, and the immune adherence receptor in this species has yet to be identified (Krych-Goldberg et al., 2001, Immunol Rev 180: 112-122). Thus, in order to evaluate the safety of and potential efficacy of our heteropolymer technology, specifically the antigen-based heteropolymers (AHP), studies in cynomolgus monkeys, a non-human primate, were performed. As a spontaneous model for SLE is not available in this species, a SLE model in which plasma from SLE patients containing IgG and IgM anti-dsDNA antibodies is infused into cynomolgus monkeys before infusion of AHP was used.

Infusion and Sampling of Monkeys

Young adult female cynomolgus monkeys were obtained from Primate Products, Inc., Miami, Fla. and housed at Therimmune Research Corporation, Gaithersburg, Md. After a 30 day acclimation period at Therimmune during which they were observed for general health and suitability and exhibited at least two negative tuberculosis tests, they were released for study use by a staff veterinarian. All study procedures were conducted according to the FDA Good Laboratory Practice Standards, 21 CFR Part 58.

Each monkey was assigned a unique number, individually housed, and identified by a chest tattoo and an ear tag and the cage was labeled with a cage card. Prior to dosing, the monkeys were fasted overnight with water available. Animals were sedated via ketamine (0.1 ml/kg) injection in the saphenous vein for approximately the first 30 to 90 min during dosing. After initial sedation, Artificial Tears Ointment was administered to the eyes, as needed, to prevent drying.

Human anti-dsDNA Ab and saline were removed from storage (2 to 8° C.) approximately 20 minutes prior to dosing. Monkeys 21208 and 21209 (Table 1) received 15 ml of Human anti-dsDNA Ab (ETI-051-49), monkeys 25949, 25950, 25952 and 25953 (Table 1) received 10 ml of Human anti-dsDNA Ab (ETI-051-49) administered at an approximate rate of 1 ml/min via intravenous infusion in the saphenous vein. Approximately 30 min after administration of Human anti-dsDNA Ab, each monkey received a single, intravenous slow bolus injection of saline (0.5 ml monkey 21208, 1.5 ml monkeys 25949 and 25950) or AHP mAb-dsDNA (0.5 ml preclinical material ET-051-45C monkey 21209, 1.5 ml cGMP AHP ETI 104 monkeys 25952 and 25953) via the saphenous vein. A board-certified veterinarian observed all dosing procedures.

Human SLE patient plasma samples having Farr titers in excess of 300 IU/ml (Feltkamp et al., 1988, Annals of the Rheumatic Diseases 47: 740-746) were pooled, clarified by filtration and stored at 4° C. The final SLE plasma pool (Human anti-dsDNA Ab, designated as ETI-051-49) was determined to have a Farr titer of 410 IU/ml before infusion into pilot study monkeys (ID nos. 21208 and 21209, respectively, Table 1) and 300 IU/ml before infusion into the 4 additional monkeys.

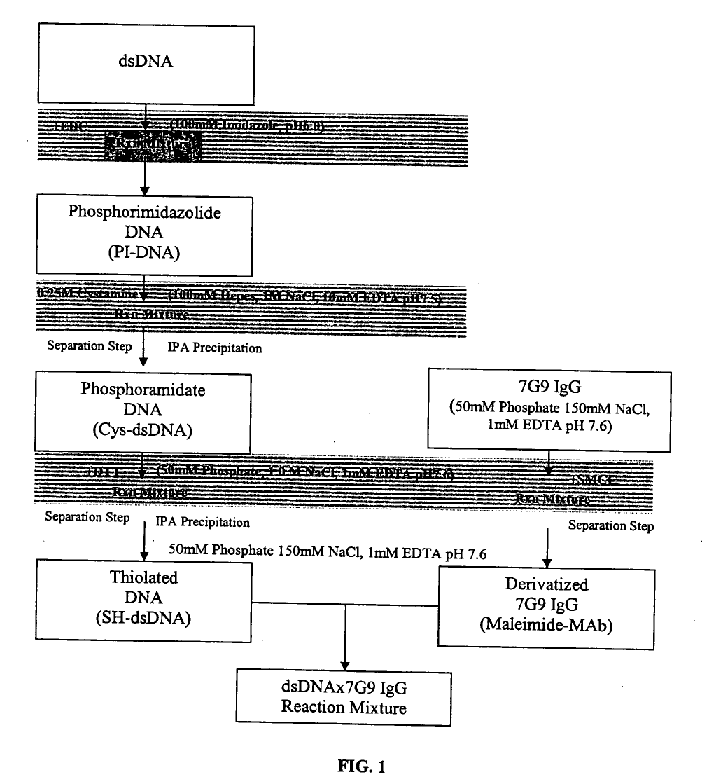

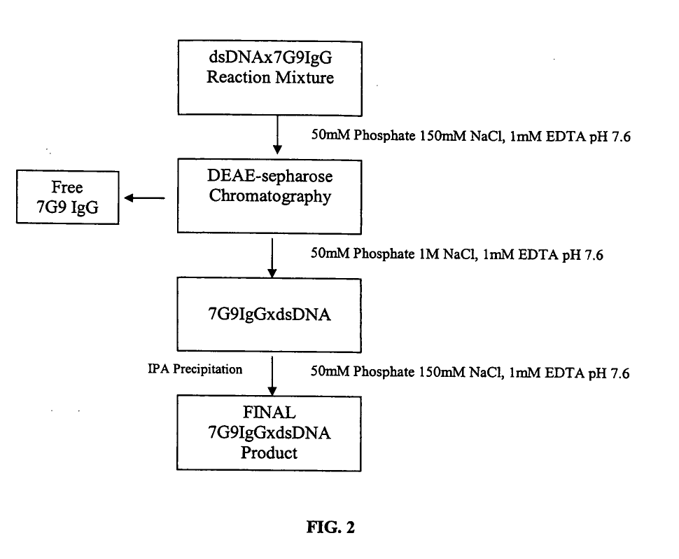

Blood samples for laboratory tests were collected from each monkey by puncture of the femoral vein. Monkey whole blood samples (1 ml) for Flow Cytometer Analysis were collected into EDTA-treated tubes (20 μl of a 0.5M EDTA solution added to each tube prior to sampling). EDTA and sodium citrate were used as anticoagulants for hematology and coagulation samples, respectively. Plasma was isolated from all designated samples by centrifugation of the EDTA coated tubes at approximately 3500 rpm and 4° C. for approximately 10 min, placed into fresh-labeled tubes, and kept frozen at or below −70° C. until analysis. All blood samples for serum isolation were collected in Vacutainer™ Serum separator tubes. Within 30-80 minutes of collection (when a clot was visible), the serum was separated from the clotted cells by centrifugation at approximately 3500 rpm and 4° C. for approximately 10 min, placed into fresh-labeled tubes, and kept frozen at or below −70° C. until analysis. Each monkey was supplemented with saline after the 2 hr blood (FIG. 1) collection interval. A maximum of 60 ml was administered subcutaneously at three different sites (approximately 20 ml per site).

The monkeys were removed from their cages and detailed physical examinations were performed. Individual body weights were recorded prior to dosing, and at the end of the test period. The monkeys were observed for mortality, morbidity and clinical signs of toxicity twice daily by cage side observation, with at least 6 hrs between observations. Prior to dosing, and at the end of the test period the dose site on monkeys 21208 and 21209 was graded using standardized evaluations of the Draize Scoring System (edema and erythema). Each monkey was given 12 biscuits divided equally between the morning and afternoon and remaining biscuits were counted prior to the next feeding. For monkeys 21208 and 21209, blood pressure, heart rate and electrocardiograms were measured at random and approximately 2 minutes after dosing.

Flow Cytometry Assays

Pico Green (Catalogue # P-7581 Molecular Probes, Eugene, Or.) was obtained as a stock solution of unspecified concentration in dimethyl sulfoxide, and diluted into BSA/PBS (Dulbecco's PBS Gibco catalogue # 21300-058 containing 1% w/v Bovine Serum Albumin Sigma catalogue # A7906) and 0.03% Sodium Azide) immediately before use. Flow cytometry probes were also obtained from Molecular Probes: Alexa 488 Goat anti-mouse IgG antibody (catalogue # A-11029), Alexa 488-labeled Goat anti-human IgG (catalogue # A-11013), and Alexa labeled Goat anti-human IgM (catalogue # A-21215).

From each whole blood sample a 10 μl aliquot was removed and placed into a fresh labeled tube containing 2 ml ice cold BSA/PBS. Samples were washed twice with 2 ml BSA/PBS and centrifugation of 3000 RPM for 3 minutes per wash. After the second wash the samples were resuspended to an ˜1% (v/v) erythrocyte concentration in BSA/PBS.

Duplicate 50 μl aliquots of 1% monkey E test samples were placed into a freshly-labeled staining tube and 50 μl of the optimum dilutions of each stain (in BSA/PBS) were added (1:200 dilution Pico Green or Alexa-anti-mouse; 25 μl 1:100 Alexa anti-human IgG plus 25 μl 1:100 Alexa anti-human IgM). Samples were incubated at 22±8° C. with shaking for 30 minutes. Samples were washed twice with 2 ml BSA/PBS and centrifugation of ˜3000 RPM for 3 minutes per wash. After the second wash the samples were resuspended in 1 ml 1% paraformaldehyde (1:4 dilution, Cytofix Pharmingen, catalogue #554655) and stored at 4° C. or on ice until assayed on the flow cytometer. To control for non-specific autofluorescence, monkey whole blood samples were processed as described for staining but incubated in BSA/PBS before fixation with paraformaldehyde.

Monkey plasma test samples were examined for the presence of human Ig (G+M) antibody against dsDNA. Whole blood from non-study animals was collected in EDTA as described above, pooled, diluted with an equal volume of Alsever's solution (Sigma catalogue #A3551) and stored at 4° C. To prepare erythrocytes, whole blood was centrifuged at ˜3000 RPM for 3 minutes, plasma and buffy coat layers were removed by aspiration. The erythrocyte pellet was washed twice with 2 ml BSA/PBS and centrifugation at ˜3000 RPM for 3 minutes per wash. After the second wash the samples were resuspended to an ˜10% (v/v) erythrocyte concentration in BSA/PBS. Erythrocytes were opsonized by adding a saturating amount of AHP (3 μg/ml) and incubating for 15 minutes at 37° C. with shaking. Samples were washed twice with 2 ml BSA/PBS and centrifugation of ˜3000 RPM for 3 minutes per wash. After the second wash, the samples were resuspended to an ˜1% (v/v) erythrocyte concentration in BSA/PBS. To 50 μl aliquots of AHP opsonized 1% monkey E, 50 μl of monkey plasma was added and the samples incubated for 15 minutes at 37° C. with shaking. After washing twice with BSA/PBS the samples were resuspended in 50 μl BSA/PBS and stained with Alexa anti-human Ig (G+M) as described above. Stained samples were analyzed on the flow cytometer. To control for non-specific binding of monkey antibody to monkey erythrocytes, aliqots of each plasma sample were incubated with non-opsonized monkey E (BSA/PBS treated) and stained with the Alexa anti-human Ig (G+M) mixture.

Quality control samples were prepared and analyzed each day along with monkey samples. AHP binding control samples were prepared by opsonizing pooled naive monkey 10% E with 10 μg/ml, 5 μg/ml, 2.5 μg/ml, 1.25 μg/ml, 0.625 μg/ml, 0.31 μg/ml or 0.16 μg/ml AHP or with BSA/PBS, washing and then staining with pico green or Alexa anti-mouse IgG. Anti-dsDNA antibody binding control samples were prepared by opsonizing naive monkey 110% E with 3 μg/ml AHP, then washing and incubating 1% (v/v) opsonized E with SLE plasma (ETI-051-49) concentrations of 80, 40, 20, 10, 5, 2.5 or 1.25 Farr IU/ml. After washing, these samples were stained with Alexa anti-human Ig (G+M) mixture. All quality control samples were resuspended in 1 ml 1% paraformaldehyde (1:4 dilution, Cytofix Pharmingen, catalogue #554655) and stored at 4° C. or on ice until assayed on the flow cytometer.

All samples were analyzed on a FACSCalibur flow cytometer using an erythrocyte gate set on size (Forward Scatter) and granularity (Side Scatter). Data (20,000 events per sample) were analyzed using Cell Quest software to determine the mean channel of fluorescence intensity.

CR1 values for the monkey E (Table 1) were determined by radioimmunoassay at saturating anti-CR1 mAb 7G9 (Ross et al., 1985, J Immunol 135: 2005-2014).

Hematology, Chemistry Coagulation and Farr Assay Laboratory Tests

The hematology and coagulation samples were stored at 2-8° C. In order to complete the glucose-6-phosphate-dehydrogenase (G6PD, parameter of red blood cell functionality) assay, an aliquot of the whole blood for the hematology sample was diluted (1:10) in deionized water, and frozen at or below −70° C. Serum chemistry samples and plasma samples for determination of high avidity anti-dsDNA Ab activity by Farr assay were also stored frozen at or below −70° C.

Results

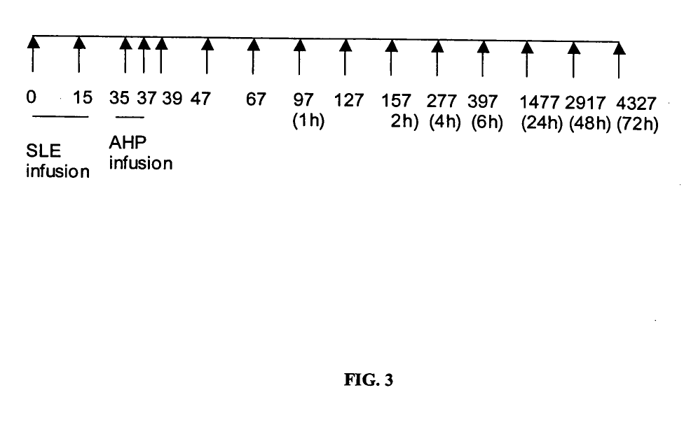

To examine the safety of AHP administration and to define initial pharmacokinetics, a pilot study was performed in two young adult female cynomolgus monkeys (21208 and 21209, Table 1). The outline of the pilot study is shown in FIG. 3 (0-24 h). Both monkeys received 15 ml of Human anti-dsDNA Ab via infusion in the saphenous vein (1 ml/min). Approximately 30 min after anti-dsDNA administration each monkey received a single, intravenous slow bolus injection of 0.5 ml saline or 0.5 mg preclinical AHP via the saphenous vein. Blood pressure, heart rate and electrocardiograms were measured at baseline (time 0) and approximately 2 minutes after dosing. Blood samples for clinical chemistry, hematology, coagulation and pharmacokinetics were collected from each monkey by puncture of the femoral vein.

There were no unscheduled deaths or clinical abnormalities seen in the monkey following a single dose of AHP and/or Human anti-dsDNA Ab. Some bruising was noted at the injection site for both monkeys. Both monkeys exhibited a minimal weight loss (0.1 g) on Day 2 as compared to Day 1, possibly due to excessive handling and blood volume loss due to blood sample collection at multiple time points. There were no apparent effects on food consumption as a result of AHP and/or Human anti-dsDNA Ab administration. Heart rate and blood pressure data recorded immediately after dosing were within the normal parameters for Cynomolgus monkeys. There were no significant electrocardiographic changes following a single dose of AHP and Human anti-dsDNA Ab. Hematology (CBC profile, hemoglobin MCV, MCH, MCHC), clinical chemistry (potassium, glucose, sodium), liver function (alkaline phosphatase, ALT, Albumin, total protein), kidney function (BUN, Creatinine, serum electrolytes), red blood cell function (G6PDH and Cholinesterase) and coagulation (prothrombin time, activated thromboplastin time) data did not reveal any changes that were related to AHP and anti-dsDNA Ab administration. Most values were comparable to the baseline values and were generally within reference ranges.

AHP binding to monkey RBC was measured using two separate FACS assays, Pico Green staining detects the binding of the salmon testis component of AHP to the RBC, while the 7G9 CR1 mAb component of AHP is detected via Alexa-conjugated anti-mouse antibody. Binding of AHP was evident on the erythrocytes of monkey 21209 within 2 minutes of receiving the drug, and could be detected for up to 24 hours after injection. Human anti-dsDNA Ab, detected as hIgG, was bound to monkey 21209 erythrocytes after AHP infusion. Plasma levels of Human anti-dsDNA Ab detected as either Farr activity or hIgG by FACS were reduced by 65-75% within 2 min of AHP infusion and remained reduced by 42-47% at 1 hr post infusion. In contrast in monkey 21208 that received no AHP, plasma levels of Human anti-dsDNA Ab were reduced by 8-17% over the same 1 hr period.

Based on the safety and pharmacokinetic profiles seen in the pilot study, a study using 4 additional cynomolgus monkeys was carried out. All four monkeys received an intravenous infusion of 10 ml SLE plasma followed approximately 30 min later by a single intravenous slow bolus injection of 1.5 ml saline (monkeys 25949 and 25950 Table 1) or 1.5 mg cGMP produced AHP (monkeys 25952 and 25953 Table 1). The monitoring of AHP pharmacokinetics was extended to 3 days post administration.

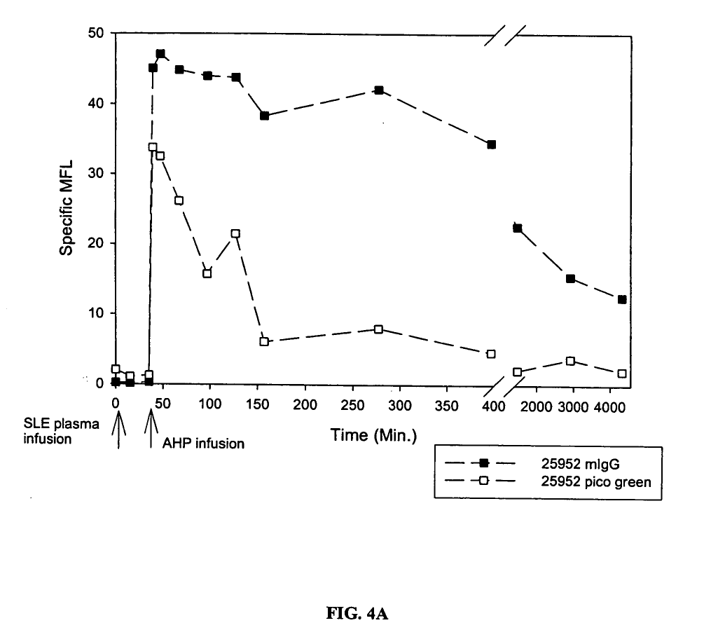

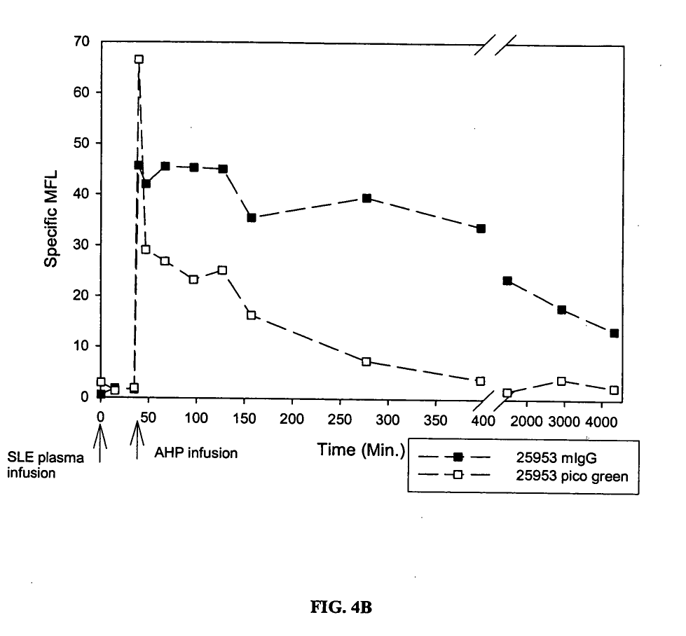

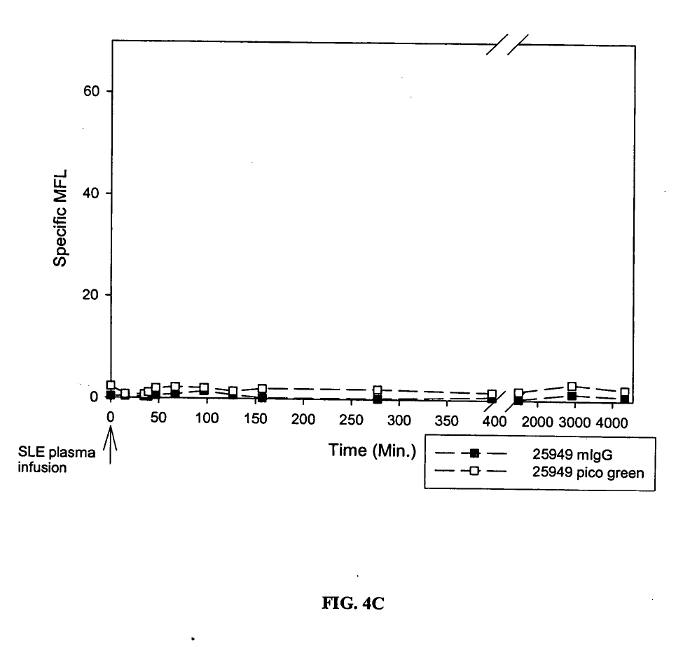

Binding of AHP to erythrocytes of monkeys 25952 and 25953 (FIGS. 4A and 4B) occurred rapidly with maximal binding detected at the earliest time point examined (2 min post infusion). As seen in FIGS. 4A and 4B, the 7G9 CR1 mAb component of AHP remains associated with monkey RBC during the first 6 h post infusion (approximately 75% of the maximum fluorescence is still present) and can still be seen associated with monkey RBC even after 3 days (30% of the maximum fluorescence). The association of the salmon testis DNA component of AHP follows similar rapid kinetics (maximal binding detected at the 2 min time point) but 90% of the peak fluorescence signal is lost during the first 6 h post infusion. As seen in FIG. 4C for monkey 25949, (similar results were observed for monkey 25950) only background fluorescence is detected via Pico Green or Alexa anti-mouse staining of monkey RBC in the absence of bound AHP.

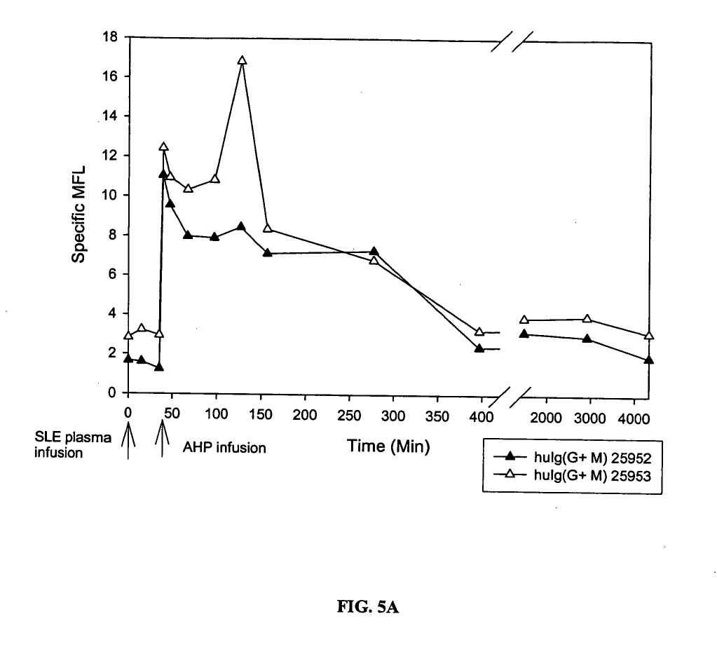

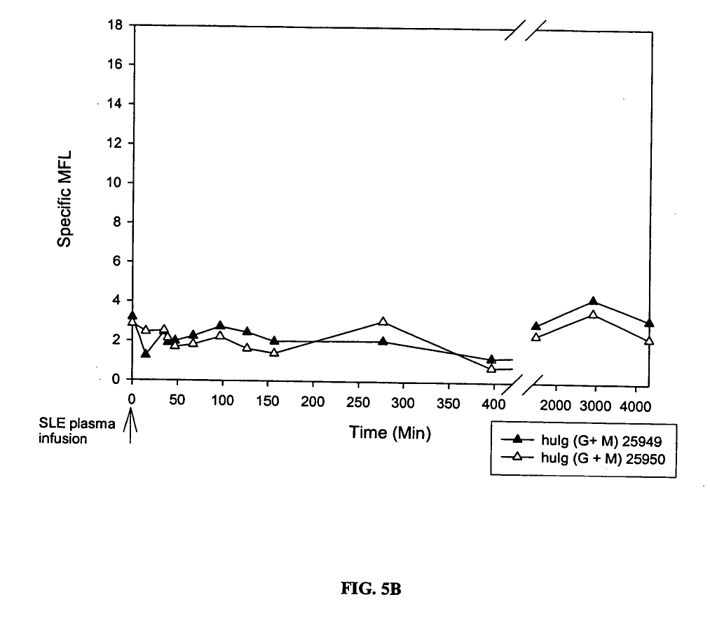

Whether Human anti-dsDNA Ab was associated with AHP bound to monkey RBC was also examined. Subsequent to the pilot experiment, it was determined that 30% of the Human anti-dsDNA Ab in the SLE plasma infused was IgM, so we stained with a mixture of Alexa anti-human IgG and IgM antibodies. As seen in FIG. 5A, huIg (G+M) binding to RBC from monkeys 25952 and 25953 was seen 2 min after AHP infusion and 50-60% of the maximum fluorescence was still seen associated with RBC at 4 h post infusion. Only background RBC fluorescence was seen in the monkeys that did not receive AHP (FIG. 5B).

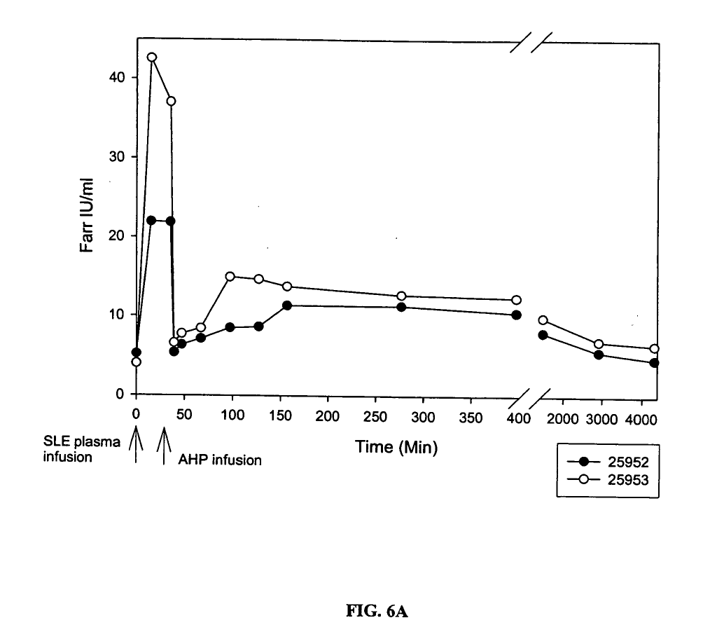

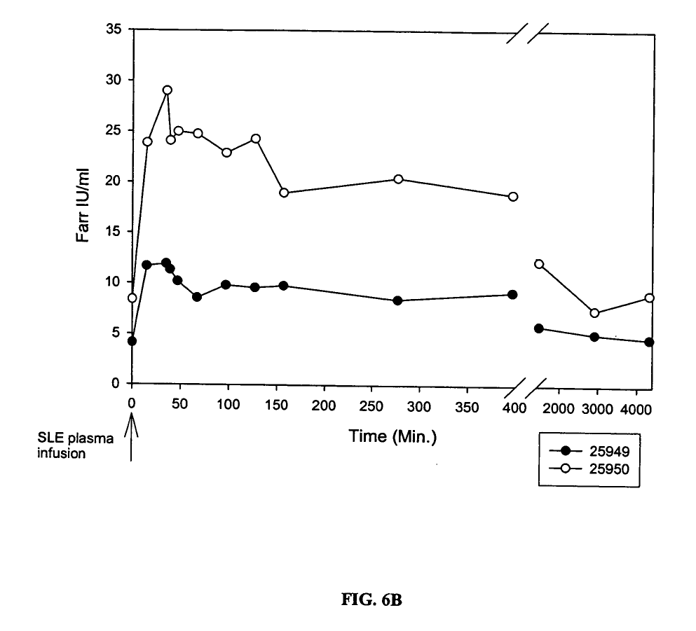

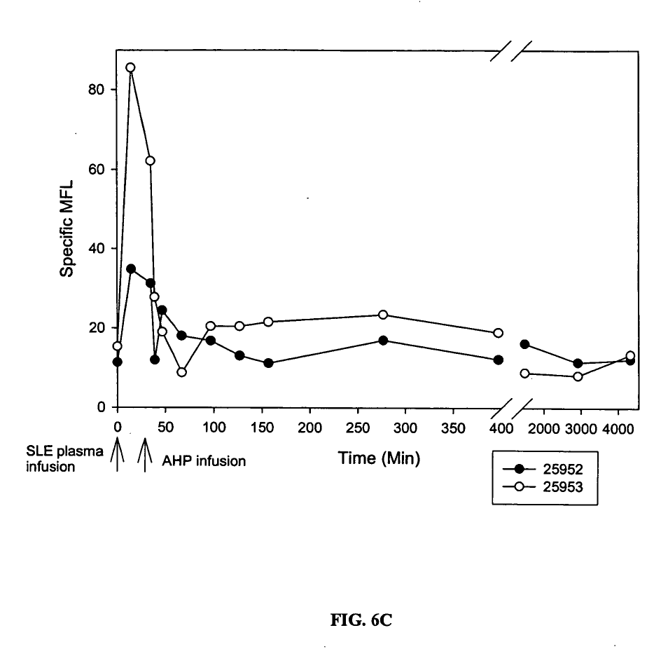

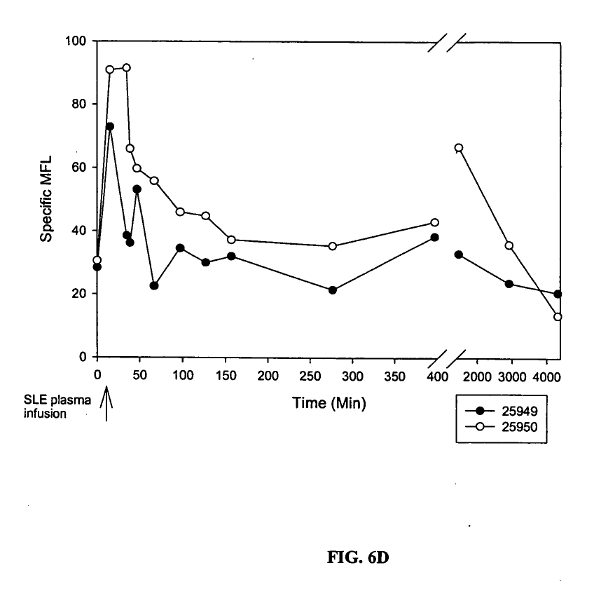

Clearance of Human anti-dsDNA Ab from the plasma of these four monkeys was also examined. The Farr assay determines the level of high affinity anti-dsDNA Ab in serum or plasma, by virtue of its ability to bind radiolabeled dsDNA and precipitation of the Ab-DNA complex by addition of saturated ammonium sulfate (final ammonium sulfate concentration of 33-50%), in comparison to a reference standard (Aarden et al., 1976, J Immunol Meth 11: 153-163; Aarden et al., 1976, J Immunol Meth 10: 39-48). As can be seen in FIG. 6A, infusion of AHP into monkeys 25952 and 25953 resulted in a rapid reduction in the plasma Farr titer (>90% at 2-30 min post infusion) which returned to 20-30% of its maximum value by 1 h post infusion. In contrast, monkeys 25949 and 25950 (no AHP group) demonstrated plasma Farr titers (FIG. 6B) that remained at 60-80% of their maximum over this same time period. As expected from the Farr results, the level of plasma Human anti-ds Ab (human Ig) in monkeys 25952 and 25953 (FIG. 6C) was rapidly reduced following AHP infusion, resulting in background fluorescence by 30 min post infusion. In monkeys that did not receive AHP (25949 and 25950) the level of plasma human anti-ds Ab (human Ig) decreased at a much slower rate over this same time period (FIG. 6D).

TABLE 1
Monkeys included in studies

| | | | | Blood | Total | Predicted | | % CR1 |
|---|---|---|---|---|---|---|---|---|
| | Monkey | | | volume^{c} | Farr | Farr Titer | AHP | sites on E |
| | # | CR1/E | Kg | (ml) | Units | (IU/ml) | (mg) | saturated |
| Study | 21208 | 3926 | 3.4 | 221 | 6150 | 27.8 | 0.0 | N/A |
| I | 21209 | 4236 | 2.8 | 182 | 6150 | 33.8 | 0.5^{a} | 5.3 |
| Study | 25949 | 459 | 3.0 | 195 | 3000 | 15.4 | 0.0 | N/A |
| II | 25950 | 725 | 3.2 | 208 | 3000 | 14.4 | 0.0 | N/A |
| | 25952 | 7721 | 2.9 | 189 | 3000 | 15.9 | 1.5^{b} | 8.4 |
| | 25953 | 3690 | 2.2 | 143 | 3000 | 21.0 | 1.5^{b} | 23.1 |

^{a}Preclinical lot of AHP
^{b}cGMP lot of AHP
^{c}Blood volume calculated based on 65 ml/kg (40)

TABLE 2
In Vitro Binding of AHP to Monkey RBC Ratio PBS/Serum^{a}

| Time | Experiment | | Experiment | | Experiment | | Mean ± | Mean ± |
|---|---|---|---|---|---|---|---|---|
| Points | 1 | Anti- | 2 | Anti | 3 | Anti- | SD Pico | SD Anti- |
| (min) | Pico Green | mouse | Pico Green | mouse | Pico Green | mouse | Green | mouse |
| 2 | 2.3 | 1.9 | 2.0 | 2.3 | 1.8 | 1.5 | 2.03 ± 0.25 | 1.90 ± 0.40 |
| 5 | 2.8 | 2.2 | 2.3 | 2.1 | 1.8 | 1.6 | 2.30 ± 0.50 | 1.97 ± 0.32 |
| 10 | 3.1 | 2.3 | 2.4 | 2.0 | 1.8 | 1.4 | 2.43 ± 0.65 | 1.90 ± 0.46 |
| 20 | 4.3 | 2.5 | 2.9 | 2.1 | 1.8 | 1.2 | 3.00 ± 1.25 | 1.93 ± 0.67 |
| 30 | 5.4 | 2.6 | 3.4 | 2.1 | 1.9 | 1.2 | 3.57 ± 1.76 | 1.97 ± 0.71 |
| 60 | 4.4 | 3.0 | 5.4 | 3.6 | 2.2 | 1.0 | 4.00 ± 1.64 | 2.53 ± 1.36 |

^{a}Ratio of mean fluorescence of monkey rbc bound to AHP in PBS to the mean fluorescence of monkey rbc bound to rbc in monkey plasma detected by staining with Pico Green or Alexa anti-mouse antibody.

In this example the effect of AHP binding to monkey E and the effect on multiple parameters that are necessary to determine the safety of a biological agent in a toxicology screen were also determined. Furthermore, the pharmacokinetics of AHP binding in vivo to monkey E and whether E-AHP complex could indeed bind to and remove dsDNA antibodies from the circulation were also determined.

Infusion of AHP into monkeys resulted in rapid binding to monkey E (maximum binding detected within the first two minutes) when detected by either staining of its dsDNA moiety with a DNA-binding fluorescent dye (Pico Green) or labeling of its CR1 mAb moiety with fluorescent anti-murine IgG (FIGS. 4A and 4B). The specificity of these assays for AHP binding was confirmed by their failure to detect fluorescence signal on E from monkeys which did not receive AHP infusion (FIG. 4C). Binding of human IgG and IgM anti-dsDNA antibodies to monkey E did not occur before AHP infusion, nor in monkeys which did not receive AHP infusion (FIG. 5B), but occurred rapidly (within 2 minutes) after AHP infusion (FIG. 5A).

The rate at which the two components of AHP (dsDNA and mAb) and human IgG/IgM anti-dsDNA antibody were cleared from monkey E did not appear as tightly coupled as their rate of association (FIGS. 4A and 4B, FIG. 5A). Thus, while the CR1 mAb component of AHP remained substantially associated with monkey E for 4-6 hrs after infusion, the dsDNA component of AHP seemed to rapidly decrease over the 4-6 hr period. Since the human IgG/IgM anti-dsDNA Ab should be bound to the dsDNA component of AHP, cleavage of the dsDNA component should result in an immediate reduction in the level of human IgG/IgM anti-dsDNA Ab associated with E. However, the association of the human IgG/IgM anti-dsDNA Ab with monkey E closely mirrored the CR1 mAB component. To determine if these results could be due to interference of the assays, the time course of binding of a fixed amount of AHP to monkey E in reactions in vitro in either BSAIPBS diluent or monkey serum was examined. The results demonstrated that Pico Green and Alexa anti-murine fluorescence signals were reduced approximately 2-fold in monkey serum compared to BSA/PBS and that the reduction in signal intensity was greatest for Pico Green (Table 2). Thus the rapid in vivo loss of the Pico Green signal may not reflect AHP degradation but may be due to an interference phenomenon or matrix effect specific to this probe.

AHP administration resulted in a rapid reduction in the plasma level of anti-dsDNA antibody as detected by Farr assay (greater than 90% reduction in 2-30 min). In contrast, plasma Farr titers fell by only 20-40% over the same time period in monkeys that did not receive AHP infusion (FIGS. 6A and 6B). That this represented clearance of the high affinity anti-dsDNA antibody activity from the circulation and not inactivation is suggested by the similar reduction in total human IgG/IgM anti-dsDNA antibody level in plasma samples from the AHP infused monkeys as measured by flow cytometry after incubation with AHP coated E's (FIG. 6C). A slow rate of clearance of human IgG/IgM anti-dsDNA antibody and Farr activity in the monkeys that did not receive AHP infusion was not unexpected as these anti-dsDNA antibodies can be complexed with DNA in SLE plasma (Williams et al., 1980, Proc Eur Transplant Assoc 17: 629-636; van Bruggen et al., 1994, Neth J Med 45: 273-279; Hylkema et al., 2000, J Autoimmun 14: 159-168, 2000) and these immune complexes would be removed through normal clearance pathways.

These results have demonstrated that the administration of AHP to these monkeys was safe. Included in the safety assessment were many parameters deemed necessary to establish a toxicity profile including cardiovascular profile, red blood cell function, basic metabolic chemistry, hematology, liver and kidney function, and observations of systemic and local reactions. The safety profile for AHP administration was extended by performing a toxicology study in which vehicle or AHP (0.5 or 1.5 mg) was administered to groups of 4 monkeys. The parameters described above were monitored through 8 days post AHP administration, at which time 2 monkeys from each group were sacrificed, organs removed, weighed and examined for gross pathological changes. Tissue sections were prepared and examined microscopically by a board-certified veterinary pathologist. The remaining 2 monkeys from each group were examined in the same manner on day 15 post AHP administration. The findings paralleled the results in the clearance study and in addition determined that there were no gross or histopathological changes due to administration of either dose of AHP. It was also determined that very low levels of primate anti-murine antibody, specifically recognizing 7G9 mAb, were elicited by a single infusion of AHP. There was neither a difference in the magnitude of the immune response to the 0.5 mg or 1.5 mg dose nor an enhanced response in the animals examined at day 15 versus day 8.

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed is:

1. A method for producing a purified composition comprising bispecific molecules, said bispecific molecules each comprising (a) an antigen recognition portion that binds a C3b-like receptor and (b) one or more double-stranded DNA molecules cross-linked to said antigen recognition portion, said method comprising precipitating said bispecific molecules using an alcohol solution.

2. The method of claim 1, wherein said alcohol solution contains 50% of isopropyl alcohol by volume.

3. The method of claim 1, wherein said antigen recognition portion that binds a C3b-like receptor is an anti-CR1 monoclonal antibody.

4. The method of claim 3, wherein said one or more double-stranded DNA molecules each has a size in the range of 100-5000 base pairs.

5. The method of claim 4, wherein said one or more double-stranded DNA molecules each has a size in the range of 200-3000 base pairs.

6. The method of claim 5, wherein said one or more double-stranded DNA molecules each has a size in the range of 500-2500 base pairs.

7. The method of claim 6, wherein said one or more double-stranded DNA molecules each has a size in the range of 500-1500 base pairs.

8. A purified composition comprising bispecific molecules, said bispecific molecules each comprising (a) an antigen recognition portion that binds a C3b-like receptor and (b) one or more double-stranded DNA molecules cross-linked to said antigen recognition portion, wherein said purified composition is produced by the method of any one of claims 1 to 7.

9. The purified composition of claim 8, wherein the DNA concentration of said purified composition is at least 1.100 mg/ml.

10. The purified composition of claim 8, wherein the protein concentration of said purified composition is at least 0.200 mg/ml.

11. The purified composition of claim 8, wherein said purified composition has a titer of at least 0.030 mg/ml as determined by ELISA for binding to immobilized CR1 receptor molecules.

12. The purified composition of claim 8, wherein said purified composition has a free IgG protein concentration of less than 0.006 mg/ml.

13. A method for producing a purified composition comprising bispecific molecules, said bispecific molecules each comprising (a) an antigen recognition portion that binds a C3b-like receptor and (b) one or more dsDNA molecules cross-linked to said antigen recognition portion, said method comprising
(i) cross-linking an antigen recognition portion that binds a C3b-like receptor with one or more dsDNA molecules to produce a composition comprising bispecific molecules comprising said antigen recognition portion cross-linked to said one or more dsDNA molecules; and
(ii) precipitating said composition using an alcohol solution, to produce said purified composition.

14. A method for producing a purified composition comprising bispecific molecules, said bispecific molecules each comprising (a) an antigen recognition portion that binds a C3b-like receptor and (b) one or more dsDNA molecules cross-linked to said antigen recognition portion, said method comprising
(i) reacting one or more dsDNA molecules with 1-Ethyl-3-(3-dimethylaminopropyl)-carbodiimide to produce activated phosphorimidazolide-dsDNA (PI-dsDNA);
(ii) reacting said PI-dsDNA with cystamine to produce cystaminated dsDNA;
(iii) reacting said cystaminated dsDNA with dithiothreitol to produce SH-dsDNA;
(iv) reacting an antigen recognition portion that binds a C3b-like receptor with sulfosuccinimidyl 4-(N-maleimidomethyl) cyclohexane-1-carboxylate to produce maleimide modified antigen recognition portion;
(v) reacting said SH-dsDNA with said maleimide modified antigen recognition portion to produce a composition comprising bispecific molecules comprising said antigen recognition portion cross-linked to said one or more dsDNA molecules; and
(vi) precipitating said composition using an alcohol solution, to produce said purified composition.

15. The method of claim 13, wherein said alcohol solution contains 50% of isopropyl alcohol by volume.

16. The method of claim 13, wherein said antigen recognition portion that binds a C3b-like receptor is an anti-CR1 monoclonal antibody.

17. The method of claim 16, wherein said one or more double-stranded DNA molecules each has a size in the range of 100-5000 base pairs.

18. The method of claim 17, wherein said one or more double-stranded DNA molecules each has a size in the range of 200-3000 base pairs.

19. The method of claim 18, wherein said one or more double-stranded DNA molecules each has a size in the range of 500-2500 base pairs.

20. The method of claim 19, wherein said one or more double-stranded DNA molecules each has a size in the range of 500-1500 base pairs.

21. A purified composition of bispecific molecules, said bispecific molecules each comprising (a) an antigen recognition portion that binds a C3b-like receptor and (b) a double-stranded DNA molecule cross-linked to said antigen recognition portion, wherein said purified composition is produced by the method of any one of claims 13 to 20.

22. The purified composition of claim 21, wherein the DNA concentration of said purified composition is at least 1.100 mg/ml.

23. The purified composition of claim 21, wherein the protein concentration of said purified composition is at least 0.200 mg/ml.

24. The purified composition of claim 21, wherein said purified composition has a titer of at least 0.030 mg/ml as determined by ELISA for binding to immobilized CR1 receptor molecules.

25. The purified composition of claim 21, wherein said purified composition has a free IgG protein concentration of less than 0.006 mg/ml.

26. The method of claim 14, further comprising before said step (vi) a step of removing free IgG antibodies from said composition produced in step (v) using ion exchange chromatography.
